(19) United States
(12) Patent Application Publication
(10) Pub. No.: US 2006/0254599 A1
(43) Pub. Date: Nov. 16, 2006

(54) Intervention techniques for post-laminectomy syndrome and other spinal disorders

(76) Inventor: Bruce H. Levin, Merion, PA (US)

Correspondence Address: DUANE MORRIS, LLP;IP DEPARTMENT, 30 SOUTH 17TH STREET, PHILADELPHIA, PA, 19103-4196, US

(21) Appl. No.: 11/186,644

(22) Filed: Jul. 20, 2005

Related U.S. Application Data
(60) Provisional application No. 60/691,659, filed on Jun. 16, 2005.
(60) Provisional application No. 60/679,588, filed on May 10, 2005.

(51) Int. Cl. A61B 19/00

(52) U.S. Cl. 128/898

(57) ABSTRACT

The invention relates to methods of relieving spinal nerve impingement disorders (SNIDs) and symptoms associated with SNIDs. The methods involve percutaneously inserting an instrument into a spinal neuroforamen to separate a spinal neural structure and a non-neural tissue. The invention further relates to methods of assessing the physical location of spinal neural structure impingement. These methods can be used in conjunction with the methods described herein or with known surgical or therapeutic methods to provide relief to subjects afflicted with a SNID.

### CROSS-REFERENCES TO RELATED APPLICATIONS

This application is entitled to priority pursuant to 35 U.S.C. § 19(e) to U.S. provisional patent application 60/691,659, which was filed on 16 Jun. 2005, and to U.S. provisional patent application 60/679,588, which was filed on 10 May 2005.

### BACKGROUND OF THE INVENTION

The invention relates generally to the field of surgical techniques.

It is known that impingement of non-neural tissues on spinal neural structures (SNSs, including both the spinal cord and the spinal nerves) can produce symptoms such as pain, numbness, and muscle weakness in body areas including or innervated by the impinged nerve. The physical location of nerve impingement can be distinct from the body location at which a symptom of the impingement is perceived. It can therefore be difficult to correlate symptomology with causation. As a result, many surgical interventions intended to relieve the symptoms fail. Furthermore, surgical interventions, whether successful or not, can result in development of scar tissue, fibroid adhesions, or other non-neural tissue structures that can impinge on SNSs, thereby complicating or worsening the disease state and its symptoms.

Epidural steroid injections, for example, have met with varying rates of success depending upon disease entity, patient, intercurrent social, economic, and psychological issues, and mode of delivery. Success rates of surgical interventions vary according to these and other variables as well. In general, further deterioration and scar formation limit long term outcome successes in many patients.

There remains a need to improve the efficacy and safety of interventional surgical techniques to optimize patient outcome. The present invention satisfies this need.

### BRIEF SUMMARY OF THE INVENTION

The invention relates to methods of relieving a spinal nerve impingement disorder (SNID) of a vertebrate such as a human. The methods comprise percutaneously inserting an instrument into a spinal neuroforamen and displacing a non-neural tissue from the spinal neural structure (SNS) sufficiently to relieve the disorder. The non-neural tissue can be moved within, expelled from, or removed from the neuroforamen. Regardless of the method, the geometric, spatial, pressure, stress, or strain relationship of the non-neural tissue and the SNS is altered sufficiently to relieve the disorder.

The invention also relates to methods of assessing the location of impingement associated with a SNID of a vertebrate. The methods comprise stimulating the body of the vertebrate at a plurality of physical locations innervated by different portions of the SNS and assessing the neuronal response to each stimulus. The location of compression can be assessed by observing decreased response by portions of the SNS distal to the location.

### DETAILED DESCRIPTION OF THE INVENTION

The invention relates to methods of relieving spinal nerve impingement disorders (SNIDs) and symptoms associated with SNIDs. The methods involve percutaneously inserting an instrument into a spinal neuroforamen to separate a spinal neural structure (SNS) and a non-neural tissue.

The methods described herein have been demonstrated to provide substantial relief to human patients who were afflicted with painful SNIDs and who did not respond well to known treatment methods.

The invention further relates to methods of assessing the physical location of SNS impingement. These methods can be used in conjunction with the methods described herein or with known surgical or therapeutic methods to provide relief to subjects afflicted with a SNID.

### DEFINITIONS

As used herein, each of the following terms has the meaning associated with it in this section.

A “spinal neural structure” (“SNS”) is a nerve, a branch of a nerve, a bundle of nerves, or an individual neuron that is present within the vertebral column of a vertebrate along at least a portion of the origin or anatomic course of the nerve, branch, bundle, or neuron. As used herein, at least the spinal cord and the 31 pairs of spinal nerves of a human are included within the scope of the term “spinal neural structure.”

A “spinal nerve impingement disorder” (“SNID”) is an abnormal physiologic or perceived condition of a vertebrate associated with one or more of contact of an SNS of the vertebrate with a non-neuronal tissue, compression of an SNS of the vertebrate by a non-neuronal tissue, and adhesion of a non-neuronal tissue to an SNS of the vertebrate. Known examples of SNIDs include bulging and herniated disks, spondyloses, spondylolistheses (slipped disks), and post-laminectomy syndrome (e.g., those associated with fibroid adhesions to spinal nerve roots or related SNSs or by scar tissue-induced alteration of normal SNS anatomy, physiology, or function).

A disorder is “relieved” if the severity of the disorder or of a symptom thereof is lessened. Reduction or elimination of pain, numbness, or muscle weakness symptomatic of a SNID are all examples of relief of the SNID.

In the context of an SNS, a first location is more distal than a second location if the first location is farther (along a neuronal path) from the spinal cord than the second location.

### DESCRIPTION

The invention relates to a method of relieving a spinal nerve impingement disorder (SNID) of a vertebrate such as a human. The method comprises inserting an instrument into a spinal neuroforamen associated with the SNID and displacing a non-neural tissue from the SNS sufficiently to relieve the disorder. The method is suitable for minimally-invasive surgical procedures, such as percutaneous insertion of one or more devices into the neuroforamen or insertion of the devices through one or more relatively small incisions.

By way of example, the methods described herein can include blunt or non-blunt dissection of a SNS and non-neural tissues using a single instrument. This can be performed under visualization or other observation of the instrument, the SNS, one or more non-neural tissues, or the borders of the neuroforamen to permit sensitive control over the procedure by the attending medical officer. This procedure effects a separation of neural and non-neural tissues which alters the spatial or physical relationship (mechanically or otherwise) and has been found to be surprisingly effective for relieving SINDs and their symptoms.

An important aspect of the methods described herein is the discovery that the methods can be performed within the neuroforamen. Preferably, the methods are performed in conjunction with a technique whereby the inserted instrument or one or more tissues in the neuroforamen (or the borders of the neuroforamen itself) can be observed (e.g., visually or neurographically) contemporaneously with performance of the methods. By way of example, an SNS in the neuroforamen can be observed by a magnetic resonance, computerized tomography, fluoroscopic, ultrasound, or other method or using an endoscope or camera attached to or contained within the inserted instrument while non-neural tissue in the foramen is manipulated with the instrument inserted therein. Electrographic methods of observing a nerve are known and can be used to monitor an SNS during performance of the methods described herein. Owing to the criticality of SNS integrity, the SNS is preferably among the tissues observed or monitored.

It was previously known that impingement of a non-neural tissue (e.g., bone or intervertebral disk material) on an SNS can result in a variety of symptoms, including axial or radicular pain, weakness, numbness, and dysesthesia. Previously known therapeutic interventions are centered around delivery of therapeutic chemicals to the site of impingement or surgical relief of the impingement, such as by surgical bulk removal of impinging non-neural tissue, fusion of vertebral joints, or some combination thereof. A shortcoming common to these prior techniques is that their efficacy relies on identification of the correct location of the SNS impingement. Direction of known therapy to another location often fails to provide significant relief to the subject.

A further shortcoming of prior surgical techniques is that they are normally performed using relative larger incisions through multiple layers of tissue (e.g., skin, muscle, and spinal lamina). Significant tissue trauma results from such incisions and currently used surgical techniques and manipulations (e.g., endoscopic procedures). General anesthesia of the subject is required for such invasive surgery (even current endoscopic methods), and this precludes the patient from providing sensory or motor feedback during the procedure. Furthermore, post-surgical developments (e.g., formation of fibroid adhesions or generation of scar tissue) can complicate or aggravate the SNID, regardless of the short-term success or failure of the surgery.

Foraminal Procedure

An important aspect of the interventional methods described herein is the discovery that they can be performed with precision within the intervertebral neuroformina with a single instrument, which can be smaller than those currently in use and without the need for a second inserted device to visualize the first. As a result, the subject experiences less trauma, lower risk of scarring, and lower incidence of recurrence of SNID symptoms. By displacing non-neural tissue from an SNS in a spinal neuroforamen, SNIDs and their symptoms can be substantially relieved. When the methods described herein are performed in a minimally-invasive percutaneous manner, the trauma and post-surgical complications associated with traditional spinal surgery can be lessened or eliminated.

The manner in which the non-neural tissue is displaced from the SNS is not critical. By way of example, it can be displaced by resection of the non-neural tissue, by pushing or pulling the non-neural tissue out of the neuroforamen, or by moving the non-neural tissue within the neuroforamen to a position at which it does not as significantly impinge upon the SNS. The non-neural tissue and the SNS can be separated by manipulating either or both tissues. That is, the non-neural tissue can be manipulated in order to displace it away from the nerve. The SNS can be manipulated in order to displace it away from the non-neural tissue. Special care should be taken not to sever or damage the SNS if it is manipulated, consistent with known precautions for surgical manipulation of nerve tissue.

One or more instruments can be introduced into the neuroforamen directly by a percutaneous route or, optionally or in addition, by another route such as through a catheter positioned in the epidural space or in the intrathecal space. When vertebral disk, scar tissue, or other material is encountered, the instrument separates the non-neural material from the nerve. This can be achieved, using a rigid or resilient surgical instrument, by downward or outward deflection or traction applied using the instrument. Such deflection or traction can move the non-neural material within the neuroforamen or force or draw it out of the foramen. Alternatively, a gouging, cutting, abrading, disrupting, ablating, or other tissue-destroying instrument can be used to remove or degrade the non-neural material, thereby facilitating its removal from the neuroforamen—either by the same or a different instrument or by suction, aspiration, or irrigation. If the non-neural material is compressible, then an expandable device (e.g., an inflatable balloon or an expandable wire mesh) can be used to compress the material and limit or prevent its impingement upon the SNS. By way of example, balloon type intra-foraminal devices can be used to displace or flatten non-neural material to gain more room intra- or peri-foraminally or to free the SNS from impingement by the material. Alternatively, a deflated balloon, or a partially-collapsed or deformable device, can be inserted into a neuroforamen, inflated (or otherwise expanded in one or more geometric planes), and drawn distally to dislodge disk fragments, scar tissue, or other materials from the SNS, the neuroforamen, or both.

As will be apparent to a skilled artisan in this field, substantially any device that lessens impingement of the non-neural material upon the SNS can be used in these methods.

If the non-neural material impinging the SNS is bony or mineralized, then a specialized instrument may be required. In particular, an instrument capable of removing such material should be used. By way of example, a drilling, abrading, or cutting instrument can be used. Preferably, an instrument of this type includes a shield between the tissue cutting or abrading portion of the device and the SNS near which it will be placed. The shield can be shaped and sized to provide protection against neurotrauma. By way of example, an instrument can have a generally cylindrical cutting head having teeth on its curved surface and contained on about one half or two thirds of its circumference within a larger, smooth, generally cylindrical shield, such that the cutting head can be engaged with the non-neural material in the neuroforamen and shielded from the SNS in the neuroforamen. Design and operation of surgical devices of this type are within the ken of the ordinarily skilled worker in this field.

The identity of the instrument used to perform these procedures is not critical, and skilled artisans will recognize that a variety of known instruments can be used. Furthermore, design of instruments adapted to perform these procedures is within the ken of the ordinary surgical instrument designer. The procedures are amenable to performance using ordinary surgical instruments, micro-machine-type (or other minimally-invasive) instruments, and robotically- or remotely-operated instruments, for example. Non-limiting examples of suitable devices include picks, needles, probes, cannulas, elevators, spatulas, spoons, separators, dissectors, hooks, awls, burnishers, rasps, curettes, drills, burrs, screws, trephines, scalpels, scissors, forceps, retractors, pliers, syringes, balloons, and the like. The instrument can also be a device adapted to deliver a tissue- or other material-destroying agent (e.g., heat, cold, electricity, abrasion, ultrasound, vibration, laser light, maser radiation, fluid pressure, gamma radiation, microwave energy, or a chemical or biochemical agent) to a selected site.

Owing to the importance of preventing SNS damage during the procedures described herein, it is preferable that those procedures be performed while observing one or more of the tissues present in or near the neuroforamen involved in the procedure. Preferably, the SNS is observed or visualized, given its importance. The devices and methods used for visualization or observation are not critical, and it is recognized that a wide variety can be suitable for use in the methods described herein.

Introduction of an instrument into the neuroforamen can be guided by any known imaging technique, such as normal or ultrafast magnetic resonance imaging, Doppler-based imaging, or computer-assisted tomography. Imaging-guided introduction can improve the effectiveness of the treatment, reduce scarring, and improve the safety margin of the procedure. Performance of a neurogram can be effective for locating the nerve root, which serves to mark the borders of the neuroforamen.

During the procedures described herein, the vertebrate can be partially awake (e.g., subject only to local anesthesia in the region of the procedure) so that the patient can describe perception of symptoms of the SNID (or relief thereof) to assist in avoiding neurotrauma. Similarly, neurophysiologic, or electrophysiological monitoring (e.g., somatosensory evoked potential, electromyography and nerve conduction velocity, or other neural tests) can be used to document improved neurologic functioning and to avoid unnecessary neurotrauma. As described in the examples herein, substantial relief from SNID symptoms could be perceived by human patients in the time periods during which the procedures were performed, permitting the attending physician to discontinue the procedure (and the attendant risk of neurotrauma) upon achievement of relief from symptoms.

The methods described herein are effective for relieving substantially any SNID characterized by contact between an SNS and a non-neural tissue or material. Examples of SNIDs that can be relieved using these methods include bulging intervertebral disks, herniated intervertebral disks, slipped disks, spondylosis, post-laminectomy syndromes, failed back syndromes, and axial and radicular pain of unexplained SNS etiology. Symptoms of these SNIDs for which relief can be obtained using the methods described herein include axial pain, radicular pain, muscle weakness, numbness, dysesthesia, and other pathophysiologic manifestations of SNS disorders.

It is recognized in the art that the term “failed back syndrome” does not describe a well-delineated physiological condition. Instead, the term is applied to patients who experience continued, different, or additional SNID or somatic, non-neuropathic symptoms following traditional surgical interventions intended to relieve the SNID. Without being bound by any particular theory of operation, it is believed that some or all of the symptoms generally attributed to failed back syndrome are instead attributable to contact, compression, or adhesion of an SNS by another tissue in response to trauma inflicted during the traditional spinal intervention. By way of example, laminectomy or other spinal surgical intervention can provoke a wound-healing response that includes formation of fibrous tissue regions that can adhere to an SNS or its root or to other tissues in such a configuration that the fibrous tissue compresses an SNS. Intraforaminal manipulation of the SNS, the fibrous tissue, an anchor of the fibrous tissue, or some combination of these, can dislodge or shift the tissues in a way that discontinues impingement of the non-neural tissue upon the SNS and permits normal nerve function. It is believed that it is this discontinuation of impingement that was experienced by patients described in the examples herein as rapid and substantial relief of SNID symptoms.

Diagnostic Methods

Many patients afflicted with a SNID are evaluated with a heavy reliance on radiological findings, MRI findings, or both. These can be overstressed as primary diagnostic modalities as opposed to patient history and subtle clues found on physical examination. For example, many patients may have true radicular complaints by history with corresponding physical findings which may not have reached classic stages and have MRI findings which document such findings as very minimal disk protrusion, or very mild disk bulging, disks which contact but do not compress nerve roots, or which only touch the thecal sac but do not cause cord compression or deformity.

The degree of mechanical contact of a disk bulge, herniation, or arthritic joint component as assessed by imaging studies is often physiologically grossly underestimated. This difficulty is compounded by often variable and irreproducible electrophysiological studies which frequently miss more central neural component involvement in favor of more peripheral etiologies. When symptoms, history, and physical exam correlate with often minor findings on imaging studies in terms of neurologic distribution, these minor findings can represent the anatomic foci of severe pathophysiology processes. Furthermore, if there are significant imaging findings in remote locations, this can be a marker of physiologic trespass at other spinal locations. This is often the case with cervicogenic headaches.

Although a cervicogenic headache may originate at the C2-3 spinal levels in a human, a herniated disk or arthritic changes at C7 level may be a marker that gravitational, stress, or other mechanical factors have effected physiologic trespass along the entire C spine, even though imaging studies are not strongly positive at C2 or C3. This may be likened to a train crash where the first few cars are severely damaged, while on gross appearance the last cars are not. However, detailed inspection of these cars on the inside would reveal some damage that is not very noticeable on cursory evaluation.

The invention includes a more effective modality of evaluation based primarily on patient complaints history, physical exam, and other circumstance wherein imaging studies are used merely as clues. This aspect of the invention relates to a method of assessing which SNS is being impinged in a vertebrate afflicted with a SNID. The method comprising stimulating a portion of the body of the vertebrate at a plurality of physical locations innervated by different SNSs in order to assess which SNS is involved in the SNID. Once an involved SNS is identified, different portions of the SNS can be stimulated to identify the approximate position of the impingement. The SNS and its impinged portion can be identified by assessing the neuronal response to the applied stimuli. SNSs and portions thereof that are affected by impingement exhibit less (or no) response to stimuli distal to the point or region of impingement.

Determination of the symptomatic nerve segment(s) can be made using the methods described herein in situations in which imaging or other studies do not fit optimally with clinical picture utilizing foraminal or nerve root challenge. An example of how this can be achieved follows.

A needle, cannula, or other delivery device is inserted into each of the suspected nerve roots or foraminal zones. Each foramen is challenged by introduction of contrast media, a chemical, or another fluid, or by application of electric, light, sound, ultrasound, radio, radiofrequency, magnetic, heat or microwave energy stimulation. This challenge can be performed with or without electrophysiological readings. By obtaining concordant complaint(s) from the patient for each such challenge, the likely spinal level of pain and or other symptom generation can be diagnosed.

Compressed or irritated nerve roots are not equally affected at all points along the nerve root or course of the nerve. For example, a disk herniation may chemically or mechanically compromise a radicular or other nerve at only one section and in one location along that section (e.g., inferiorly). Desirable effects of medication or intervention are enhanced by placement or intervention along the location of greatest trespass. The precise location of greatest trespass is located by challenging the nerve as above in different locations along the nerve, (e.g., along three orthogonal axes about the nerve). Therapeutic interventions are directed towards those levels which are most concordant, and they may administered in series or in parallel.

Using any of the imaging techniques described herein (or, less preferably, without the aid of imaging), a trocar, endoscope, catheter, steerable catheter, grasping or dissecting mechanism, an emitter of laser or other light, ultrasound, microwave, radiofrequency, heat, or cold, or another nerve challenging device can be inserted percutaneously and used to challenge a nerve root or a nerve along its course. The nerve challenging device can, for example, be a needle or introducer, and can be asymmetric along one or more of its axes. The device can be constructed of a material which is X-ray lucent or opaque, with or without radiological marking. Materials used in devices suitable for MRI can be non-ferrous, or can be constructed of polymer, glass, carbon, nanotubules, or other substances to minimize scatter and distortion, and to optimize viewing and to avoid magnetic issues. Instruments used in conjunction with CT should be made from material likewise selected to reduce scatter. The device preferably has one or more viewable markings to assist determination of the location and orientation of the device in the patient.

Pharmaceutical Treatment

A variety of pharmaceutically active agents are known to be effective for treatment of SNIDs. However, such an agent can exhibit efficacy only if administered to a location and in such a manner that the agent is able to exert its pharmacological effect on a relevant biological structure—that is, a tissue involved in the SNID. As described herein, prior methods of treating SNIDs are hampered by difficulty identifying relevant SNSs and the location of SNS impingement.

The diagnostic methods described herein for identifying an SNS involved in a SNID and for identifying the location of an impingement on that SNS can be used to direct administration of known SNID-relieving agents to body locations at which the agents can be effective. The invention includes methods of treatment comprising epidural injection of steroid, anti-neuropathic, and anti-inflammatory compounds alone or in combination for back pain, disk disease, facet disease, spondylosis, failed back/neck syndrome, radiculopathy, radiculitis, radicular symptoms, spinal stenosis, headache, migraine, cluster headache, and related disorders, including the SNIDs described herein.

Treatment can be effected by delivery of agents alone or by administration of depot formulations. Depot formulations can be made using micelles or liposomes, which can be composed or formulated with anti-inflammatory lipids or fatty acids (e.g., cetyl myristoleate) or with standard compounds, such as polyethylene glycol or related compounds, and/or other membrane stabilizing agents. Such formulations are known in the art

Examples of such agents which can be thus administered include anti-neuropathic agents, including all anti-seizure medications, such as KEPPRA®, LAMICATAL®, TOPAMAX®, TIAGABINE®, TRILEPTAL®, ZONEGRAN®, TEGRETOL®, DILANTIN®, DEPAKOTE®, NEURONTIN®, CYMBALTA®, GABATRIL®, pregabalin, carbemazapine, oxcarbazine, tricyclic antidepressants, serotonin inhibitors, ketamine and other NMDA antagonists, NSAIDs, COX2 inhibitors, calcium, sodium, potassium, chloride inhibitors, depot local anesthetics, and polyethylene glycol.

Other examples of agents which can be thus administered include anti-inflammatory agents, including TNF antagonists such as etanercept (ENBREL®, Immunex Corporation); infliximab (REMICADE®, Johnson and Johnson); D2E7, a human anti-TNF monoclonal antibody (Knoll Pharmaceuticals, Abbott Laboratories); CDP 571 (a humanized anti-TNF IgG4 antibody); CDP 870 (an anti-TNF alpha humanized monoclonal antibody fragment), both from Celltech; soluble TNF receptor Type I (Amgen); pegylated soluble TNF receptor Type I (PEGs TNF-R1) (Amgen); and a molecule containing at least one soluble TNF receptor.

Such agents can also include antagonists of one or more of the following: interleukin-1 (IL-1), IL-6, TNF-alpha, TGF-Beta; agonists of one or more of the following: IL-4, IL-10, and IL-13 agonists; and antagonists of one or more of the following: LIF, IFN-gamma, OSM, CNTF, TGF-beta, GM-CSF, IL-11, IL-12, IL-17, IL-18, IL-8 tachykinins, VIP (vasoactive intestinal peptide), and VPF (vascular permeability factor), caspase-1, caspase-5, PYCARD, NALP1, the SIS family of cytokines, the SIG family of cytokines, the SCY family of cytokines, the platelet factor-4 superfamily of intercrines, and prostaglandins. All Dosing units are as per standard dosing regimens. The agent can also be a CPLA2 inhibitor, for example.

Pressure characteristics of lesser compliance suggest spinal stenosis or local compression by scar, disk or other material. Such characteristics can also be used to direct administration.

A transforaminal administration approach can also be used, with X-ray, fluoroscopic, ultrasound, CT scanning, MRI, or fast MRI methods used to guide administration, with or without contrast.

Expandable Artificial Intervertebral Disk Matrices

Compression of nerves or nerve roots can sometimes be alleviated by increasing separation of bones (e.g., vertebrae) impinging on the nerve or its root. Surgical removal or impinging bone (e.g., laminectomy) is known. However, methods of relieving bone or bone-induced impingement on nerves have been discovered.

In one embodiment, insertion of a swellable matrix between bones (or between different processes of a single bone) and subsequent swelling of the matrix can force the bones (or processes) apart, relieving the impingement. The matrix can be made of a material that remains in a compact state during insertion or installation and thereafter expands. The expansion can be induced by absorption of water, reaction of the matrix with water, absorption of an applied solvent, reaction of the matrix with an applied solvent, application of heat to the matrix, or some combination of these, for example. Numerous materials exhibiting such swelling properties, and selection of a swellable material suitable for residence in a human body for a period of time (e.g., hours, days, months, or years) effective to relieve a disorder associated with bone or bone-induced nerve impingement is within the level of skill of an ordinary practitioner in this field.

In another embodiment, a device which can be made to expand along an axis extending between two bones or two bone processes can be inserted therein and expanded. By way of example, the device can be a cylindrical device (e.g., having a diameter of about 1 centimeter) in which separation of the circular heads of the device can be increased by rotation of a threaded screw which extends into the device from the side of the device. Such a device can be inserted and expanded to relieve bone or bone-induced impingement on a nerve or its root. Alternatively, the heads of the device can be expanded along the axis extending between the bones or processes by injecting material (e.g., an oil, water, air or other fluid) into an expandable chamber encased within the device, thereby urging the heads outwardly from the central portion of the device. The device can exhibit resilience along the axis between the bones or processes, thereby enhancing comfort and providing a more natural feel to the affected body area. By way of example, a device having two opposed flat faces having a gas-filled bladder therebetween can be inserted intervertebrally. One or both faces of the device can be attached to, or adapted for ingrowth of bone matrix from, a bone against which the face is opposed. Design and fabrication of such devices are within the level of ordinary skill for a practitioner in this field, in view of the guidance provided herein.

Furthermore, a variety of intervertebral disk replacement devices are known and described in the literature. Substantially any of these devices can be used as described herein. Selection of an appropriate physiological location at which to implant such a device can be performed using the diagnostic methods described herein.

### EXAMPLES

The invention is now described with reference to the following Examples. These Examples are provided for the purpose of illustration only, and the invention is not limited to these Examples, but rather encompasses all variations which are evident as a result of the teaching provided herein.

### Example 1

Relief of Lumbar Back Pain

A woman with failed back syndrome did not respond to a prior trans-foraminal epidural steroid injection at L5S1 although a concordant neurogram was obtained. The next time, the needle was directed to the inferior portion of the same nerve root and challenge was more concordant. Injection of steroid at this locus was very effective in relieving her radicular symptoms.

It is also notable that in contrast to common belief, neural challenge occasionally reveals that symptoms are related to a different nerve root than is suggested by dermatomal charts. For example, a patient with radiculopathy and a large disk at L45 had symptoms of thigh pain usually referable to L34, but challenge of L45 revealed concordance at this level and not at the level of L34. The patient responded to L45 injections but not to injections at L34.

In another instance, a male patient had knee symptoms with a significant L45 disk, but had symptoms that were actually from a very minimal degree of foraminal stenosis on the same side at L34, which was more concordant on challenge that L45.

### Example 2

Herniated or Bulging Disk into the Neuroforamen

A middle aged woman had suffered from left-greater-that-right radicular symptoms including pain, numbness and weakness for over 2 years secondary to a disk bulge. She had only 10-15% relief from a targeted transforaminal epidural steroid injection at the appropriate level. During that procedure, disk material was felt to be in foramen on needle placement. Therefore, the patient was taken to the procedure suite 2 weeks later.

Usual technique was followed. A 22 gauge spinal needle was placed intra-foraminally and a neurogram was obtained. A 17 gauge Tuohey needle was then introduced at the inferior portion of the neuroforamen according to the neurogram. With its bevel away from the nerve root, the disk was distracted out of the neuroforamen accompanied by a peeling sensation noted by the physician. Concurrent with the peeling sensation the patient noted that her sensation was returning to normal and her pain had decreased significantly. At that time her motor strength returned. Steroids were administered in the usual fashion, and the patients symptoms were 95% improved on that side.

### Example 3

Post-Laminectomy Syndrome

A middle aged woman with debilitating sciatica following back surgery did not respond to transforaminal epidural steroid injections. MRI showed mild scar tissue encasing the L5 nerve root. The usual transforaminal neurogram was obtained with pressure noted on injection attempt and little spread noted. The needle was advanced in several planes to shave the scar tissue from the nerve. Slight pressure was the applied to the syringe with the patient monitored for neurotrauma symptoms. Pressure was increased and a breakthrough loss of resistance noted, followed by an improved neurogram with good spread. The patient noted her symptoms improved 80%.

Follow up MRI one week later documented a significant decrease in perineural scarring and the neuroforamen was now much more patent. The procedure was repeated this time using a Tuohey needle to scrape the foramen and her symptoms of residual buttock pain disappeared. She has done well up to several months follow up. Hence this technique represents a significant alternative to risky repeat surgery. The same technique was used to decompress scar tissue with areas of cystic changes in another post-laminectomy patient and in patients with fact cysts.

### Example 4

Spinal Neuroforaminoplasty

A 60 year old man had previously undergone back surgery and continued to be afflicted with from severe axial back pain. He was deemed a poor surgical candidate, and was declined for further procedures by several neurosurgeons. The patient required high dose narcotic medication, but this reduced pain only minimally.

The patient was assessed and it was determined his symptoms were predominantly attributable to the spinal nerve of the left L45 neuroforamen. The patient was taken to the operating room and, under fluoroscopic guidance, a #17 gauge Tuohey type needle was introduced into the lateral aspect of that foramen. Foraminoplasty was performed by blunt dissection of non-neural tissue from the nerve in the foramen, and the patient noted a decrease in his pain. The patient left experiencing little pain.

The patient returned after one week with pain located lower down on the same side which was perceived as about 50% as intense as his earlier pain had been. The patient's impression was that this pain had been present earlier, but had likely been masked by the earlier more severe pain. Because his MRI showed some scar tissue in the lower L5S1 ipsilateral neuroforamen, a similar procedure was done after two weeks at that level. The patient's perception of pain decreased by about 90% and he remained well during follow up.

### Example 5

A middle-aged woman had undergone two prior laminectomies and was treated with transforaminal epidural injections on her left side. She did well until she was involved in a motor vehicle accident. Thereafter, she developed severe leg pain and low back pain and soon experienced numbness in her leg and weakness, both of which progressed to a very significant level. The patient soon developed severe radiating leg pain and allodynia which persisted. She underwent discography but was perceived to be a poor surgical candidate.

The patient underwent the foramenoplasty procedure at the L45 and L51 levels on the left side. Inserting the #17 Tuohey needle into her neuroforamina was accompanied by a surprising and immediate decrease in her pain and numbness, and a remarkable normalization of her motor strength. She did well during follow up.

The disclosure of every patent, patent application, and publication cited herein is hereby incorporated herein by reference in its entirety.

While this invention has been disclosed with reference to specific embodiments, it is apparent that other embodiments and variations of this invention can be devised by others skilled in the art without departing from the true spirit and scope of the invention. The appended claims include all such embodiments and equivalent variations.

1. A method of relieving a spinal nerve impingement disorder of a vertebrate, the method comprising percutaneously inserting an instrument into a spinal neuroforamen and displacing a non-neural tissue from the spinal neural structure (SNS) sufficiently to relieve the disorder.

2. The method of claim 1, wherein the non-neural tissue and the SNS are adhered prior to displacing the non-neural tissue.

3. The method of claim 2, wherein the non-neural tissue and the SNS are not adhered after displacing the non-neural tissue.

4. The method of claim 1, wherein at least a portion of the non-neural tissue is removed from the vertebrate.

5. The method of claim 1, wherein at least a portion of the non-neural tissue is displaced from within the neuroforamen.

6. The method of claim 1, wherein the instrument has a distal end adapted to fit within the neuroforamen.

7-9. (canceled)

10. The method of claim 1, wherein the non-neural tissue is displaced by ablating a portion of the non-neural tissue using the instrument.

11. (canceled)

12. The method of claim 1, wherein the non-neural tissue is displaced by pressure applied by a fluid supplied to the neuroforamen using the instrument.

13. The method of claim 1, wherein the non-neural tissue is a tissue other than bone.

14. The method of claim 1, wherein the non-neural tissue is intervertebral disk.

15. The method of claim 1, wherein the non-neural tissue is selected from the group consisting of spinal lamina, bone, ligament, cartilage, and fibrous adhesion.

16. The method of claim 1, wherein the non-neural tissue is scar tissue.

17. The method of claim 1, further comprising monitoring neuronal function of the SNS while inserting the instrument.

18. The method of claim 1, further comprising monitoring neuronal function of the SNS while displacing the non-neuronal tissue.

19. The method of claim 1, further comprising visualizing a tissue in the vicinity of the neuroforamen while displacing the non-neuronal tissue.

20. (canceled)

21. The method of claim 1, further comprising visualizing a tissue in the vicinity of the neuroforamen while inserting the instrument.

22. The method of claim 21, wherein at least one of the SNS, a blood vessel, a bone, and an intervertebral disk tissue is visualized while inserting the instrument.

23. The method of claim 1, comprising alternately gauging relief of the disorder and displacing the non-neuronal tissue.

24. (canceled)

25. The method of claim 1, wherein the vertebrate is a human.

26. A method of relieving a spinal nerve impingement disorder of a vertebrate, the method comprising percutaneously inserting an instrument into a spinal neuroforamen and altering the geometric arrangement of a non-neural tissue and the spinal neural structure (SNS) sufficiently to relieve the disorder.

27. (canceled)

28. A method of assessing the location of impingement associated with a spinal nerve impingement disorder of a vertebrate, the method comprising stimulating the body of the vertebrate at a plurality of physical locations innervated by different portions of the SNS and assessing the neuronal response to each stimulus, whereby the location of compression can be assessed by decreased response by portions of the SNS distal to the location.

29-35. (canceled)
